(19) United States
(12) Patent Application Publication
(10) Pub. No.: US 2026/0077082 A1
(43) Pub. Date: Mar. 19, 2026

(54) INTEGRATED AROMA DIFFUSER

(71) Applicant: Guangdong Grasse Environmental Technology Co.,Ltd., Guangzhou (CN)

(72) Inventor: Jun XIE, Guangzhou (CN)

(73) Assignee: Guangdong Grasse Environmental Technology Co.,Ltd., Guangzhou (CN)

(21) Appl. No.: 19/398,111

(22) Filed: Nov. 24, 2025

Related U.S. Application Data
(63) Continuation of application No. PCT/CN2023/120309, filed on Sep. 21, 2023.

(30) Foreign Application Priority Data
Jun. 1, 2023 (CN) 2023213786939

(51) Int. Cl. A61L 9/14

(52) U.S. Cl. CPC A61L 9/14; A61L 2209/133; A61L 2209/135

(57) ABSTRACT

The present application discloses an integrated aroma diffuser comprising a liftable aroma diffuser body slidably disposed within a guide sleeve and a lifting drive mechanism configured to drive the liftable aroma diffuser body to move vertically relative to the guide sleeve. The lifting drive mechanism achieves vertical motion of the liftable aroma diffuser body through the engagement of a gear and a rack, such that the body is sleeved within and slides up and down relative to the guide sleeve. When the liftable aroma diffuser body ascends a certain distance relative to the guide sleeve, internal components such as the essential oil bottle are exposed, thereby facilitating convenient replacement of the essential oil bottle.

### CROSS-REFERENCE TO RELATED APPLICATIONS

This application is a National Stage entry under 35 U.S. C. § 371 of International Application No. PCT/CN2023/120309, filed on Sep. 21, 2023, which claims the benefit of priority to Chinese Patent Application NO. 2023213786939, filed on Jun. 1, 2023. The entire contents of the above application are hereby incorporated by reference in their entirety.

### TECHNICAL FIELD

The present application relates to the technical field of aroma diffusers, and in particular, to an integrated aroma diffuser.

### BACKGROUND

An aroma diffuser is equipped with an essential oil bottle containing essential oil. After a period of use, the essential oil inside the bottle will be depleted, making it necessary to either replace the bottle or refill it with essential oil. Therefore, the overall structure of the aroma diffuser should be simple so that the replacement of the essential oil bottle can be performed quickly and conveniently.

A prior art aroma diffuser adopts a segmented structure. The main body and the cover of the essential oil bottle are connected by means of threads or buckles. However, the manufacture of threaded or buckle structures is time-consuming and labor-intensive, which consequently leads to a high production cost of the aroma diffuser.

Moreover, since the power supply for the air pump inside the aroma diffuser is usually disposed at the bottom of the main body, while the air pump itself is arranged on the cover, when the aroma diffuser is configured as a segmented structure, it is necessary to consider the electrical connection between the main body and the cover. In the prior art, a conductive pin insertion structure is commonly adopted, which undoubtedly further increases the overall structural complexity and production cost.

### SUMMARY

The purpose of the present application is to provide an integrated aroma diffuser with a simple structure and convenient operation, enabling the essential oil bottle to be easily replaced, thereby providing great convenience for users.

The integrated aroma diffuser comprises a liftable aroma diffuser body slidably disposed within a guide sleeve, and a lifting drive mechanism configured to drive the liftable aroma diffuser body to move vertically relative to the guide sleeve. The lifting drive mechanism achieves the driving function for the liftable aroma diffuser body through the engagement between a gear and a rack, so that the liftable aroma diffuser body is sleeved within the guide sleeve and can slide vertically relative to the guide sleeve. When the liftable aroma diffuser body ascends a certain distance relative to the guide sleeve, components inside the liftable aroma diffuser body, such as the essential oil bottle, are exposed from the guide sleeve, thereby facilitating convenient replacement of the essential oil bottle.

The lifting drive mechanism includes a mounting frame, a transmission module disposed on the mounting frame, and a lifting platform driven by the transmission module. The mounting frame includes a mounting plate, supporting columns provided around the periphery of the mounting plate, and a support plate disposed at the top ends of the supporting columns.

The transmission module comprises a motor disposed on the mounting plate and having an output shaft, a worm mounted on the output shaft, a first transmission gear disposed on the mounting plate and engaged with the worm, a first coaxial gear disposed on the first transmission gear, a second transmission gear disposed on the mounting plate and engaged with the first coaxial gear, a second coaxial gear disposed on the second transmission gear, a third transmission gear disposed on the mounting plate and engaged with the second coaxial gear, a third coaxial gear disposed on the third transmission gear, and a rack engaged with the third coaxial gear and arranged vertically.

The liftable aroma diffuser body is configured to atomize and spray essential oil into the surrounding environment. It comprises a sliding cylinder that is slidably arranged within the guide sleeve, and an atomizing module disposed within the sliding cylinder. The atomizing module comprises an essential oil bottle, an oil tube inserted into the essential oil bottle, an atomizing core body installed at the mouth of the essential oil bottle, an atomizing core disposed inside the atomizing core body and in fluid communication with the oil tube, an atomizing head sleeved over the atomizing core body, an atomizing head lower cover disposed on the top of the atomizing head, an oil baffle lower cover and an oil baffle upper cover that are engaged with each other and disposed on the atomizing head lower cover, and an atomizing head upper cover disposed on the oil baffle upper cover.

Compared with the prior art, the beneficial effects of this application are as follows:

In the present application, the atomizing module is built into a sliding cylinder that is slidably disposed within the guide sleeve. The sliding cylinder and the atomizing module inside it are driven upward relative to the guide sleeve by the lifting drive mechanism, thereby conveniently moving the essential oil bottle upward until it is exposed from the guide sleeve, enabling quick and easy replacement of the essential oil bottle. After replacement, the lifting drive mechanism drives the sliding cylinder and the atomizing module downward to retract them back into the guide sleeve, thereby completing the replacement of the essential oil bottle.

### BRIEF DESCRIPTION OF THE DRAWINGS

FIG. 1 is a perspective view of an integrated aroma diffuser of the present invention.

FIG. 2 is a partial perspective view of the aroma diffuser shown in FIG. 1.

FIG. 3a is a perspective view of the aroma diffuser of FIG. 1 viewed from another angle.

FIG. 3b is an enlarged view of part A in FIG. 3a.

FIG. 4a is an exploded perspective view showing the internal structure of the aroma diffuser of FIG. 1.

FIG. 4b is an enlarged view of a lifting drive mechanism of the integrated aroma diffuser of FIG. 4a.

FIG. 5 is a perspective view of a mounting frame of the integrated aroma diffuser of FIGS. 1-4b.

FIG. 6a is a perspective view of an oil baffle upper cover of the integrated aroma diffuser of FIGS. 1-4b.

FIG. 6b is a partial three-dimensional cross-sectional view of the oil baffle upper cover and the oil baffle lower cover of the integrated aroma diffuser shown in FIGS. 1-4b after assembly;

FIG. 7a shows a three-dimensional structural diagram of an atomizing module of the integrated aroma diffuser shown in FIGS. 1-4b.

FIG. 7b is a three-dimensional structural diagram of the atomizing module shown in FIG. 7a from another angle.

FIG. 7c is a three-dimensional structural diagram of a slide tube installed in conjunction with the atomizing module shown in FIGS. 7a-7b.

FIG. 7d shows a three-dimensional structural diagram of an essential oil level detection circuit board of the integrated aroma diffuser.

FIG. 7e also shows a three-dimensional structural diagram of the essential oil level detection circuit board of the integrated aroma diffuser.

FIG. 8a shows a three-dimensional structure of the atomizing head cover of an integrated aroma diffuser according to another embodiment of the present application.

FIG. 8b shows a three-dimensional structure of an integrated aroma diffuser according to another embodiment of the present application, illustrating the detailed structure of the oil baffle lower cover.

FIG. 9 is a sectional view showing the fit between the atomizing head upper cover and the oil baffle lower cover.

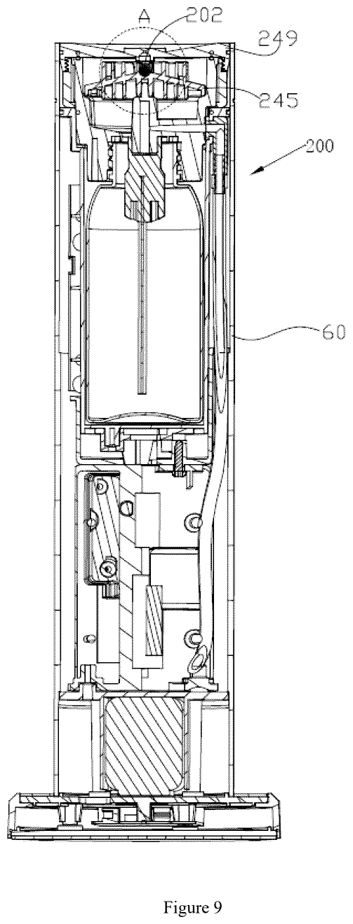

FIG. 10a shows a perspective view of the atomizing head of an integrated aroma diffuser according to another embodiment of this application.

FIG. 10b shows a perspective view of the atomizing head shown in FIG. 10a from another angle;

FIG. 11 shows a perspective view of the essential oil bottle of an integrated aroma diffuser according to another embodiment of this application.

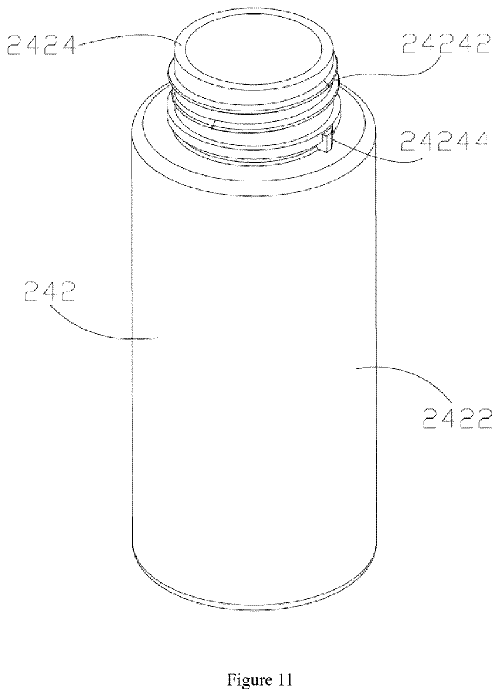

FIG. 12 shows a partial enlarged view of part A of the structure shown in FIG. 9.

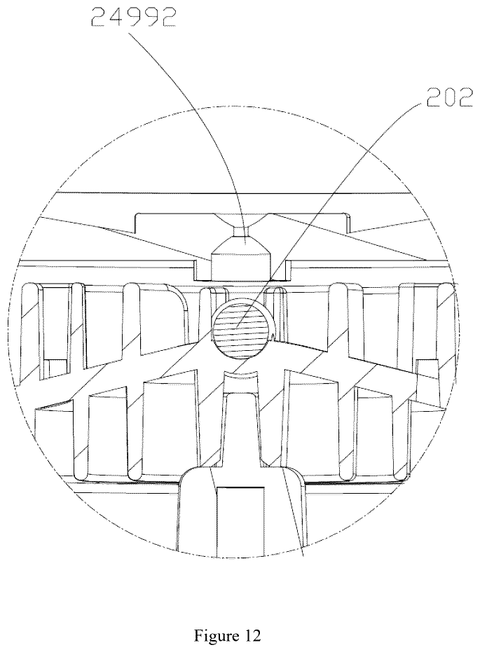

### DETAILED DESCRIPTION OF THE INVENTION

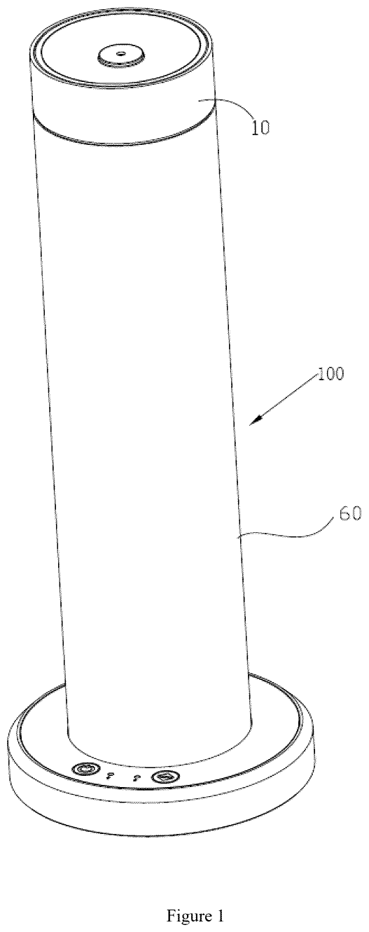

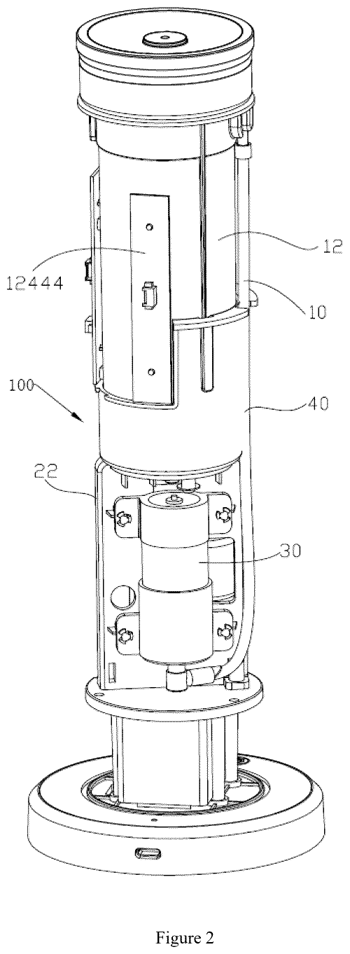

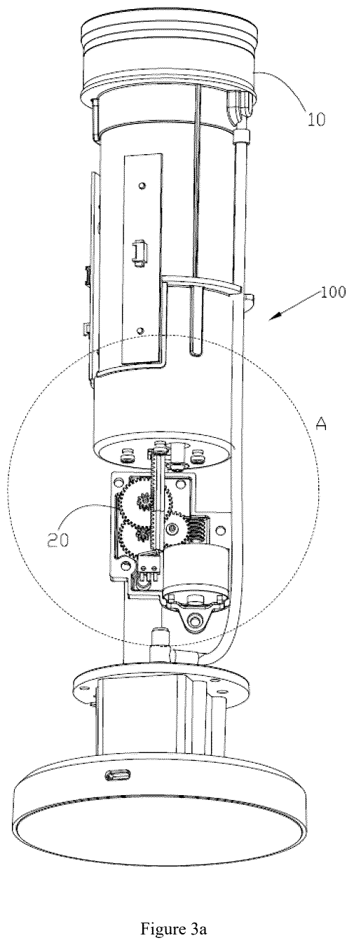

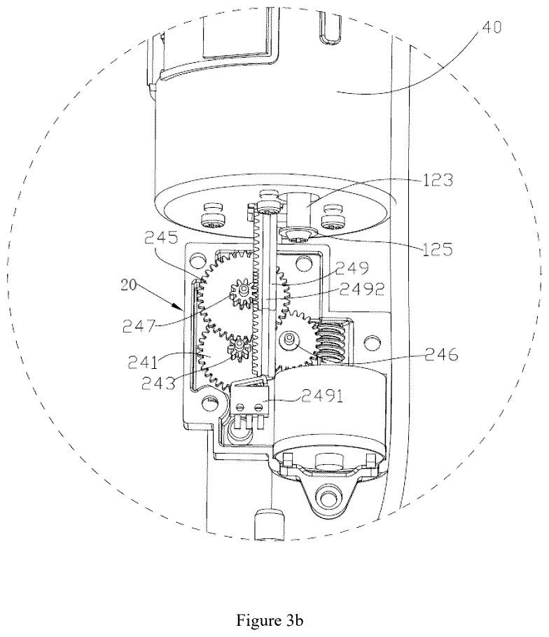

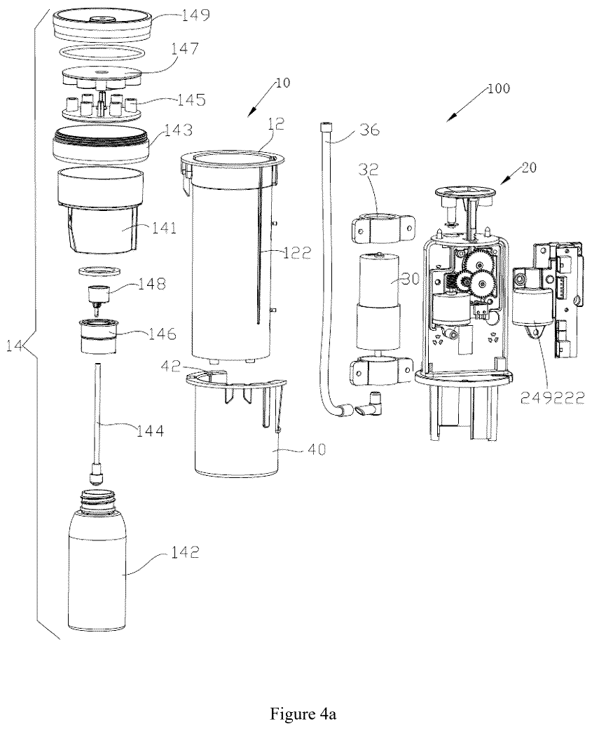

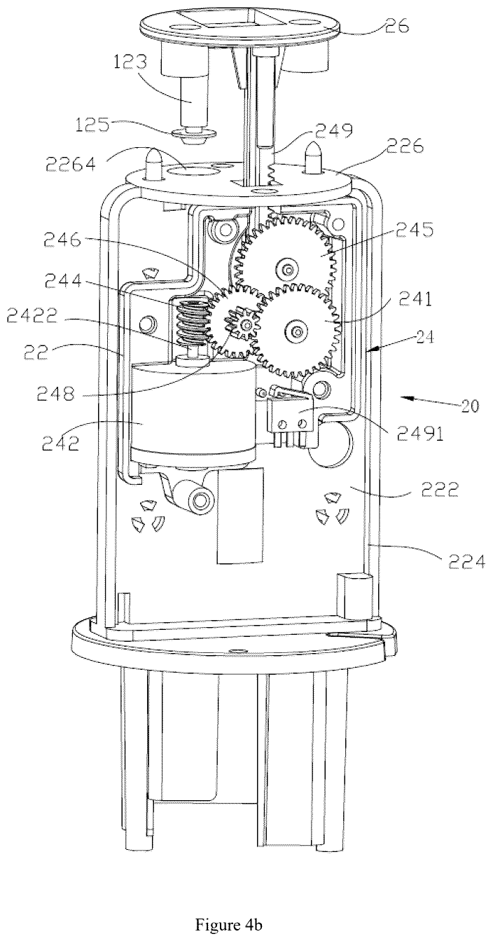

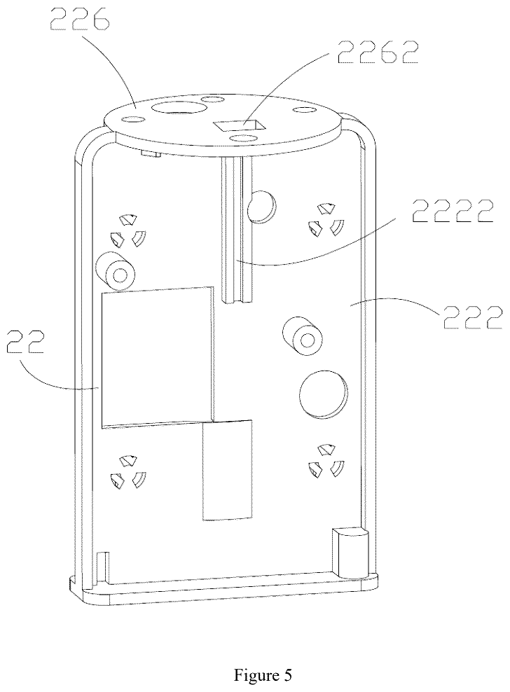

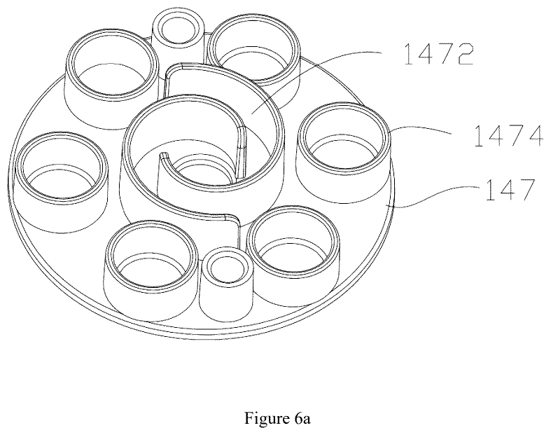

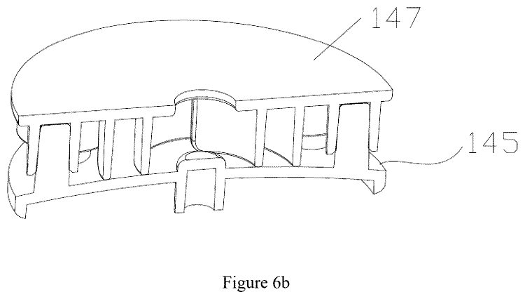

According to one embodiment of this application, referring to FIGS. 1-7e, an integrated aroma diffuser 100 includes: a liftable aroma diffuser body 10 slidably disposed within a guide sleeve 40, and a lifting drive device 20 for driving the liftable aroma diffuser body 10 to slide up and down relative to the guide sleeve 40. The lifting drive device 20 drives the lifting aroma diffuser body 10 up and down through the cooperation of a gear and a rack, ultimately rendering the lifting aroma diffuser body 10 to be fitted inside the guide sleeve 40 and slide up and down relative to the guide sleeve 40. When the liftable aroma diffuser body 10 rises to a certain distance relative to the guide sleeve 40, component inside the liftable aroma diffuser body 10, such as an essential oil bottle, is exposed from the guide sleeve 40, making it convenient for users to replace the essential oil bottle. The lifting drive mechanism 20 comprises a mounting frame 22, a transmission module 24 disposed on the mounting frame 22, and a lifting platform 26 driven by the transmission module 24. The mounting frame 22 includes a mounting plate 222, supporting columns 224 arranged around the periphery of the mounting plate 222, and a support plate 226 disposed at the top ends of the supporting columns 224.

The transmission module 24 comprises: a motor 242 disposed on the mounting plate 222 and having an output shaft 2422; a worm 244 mounted on the output shaft 2422; a first transmission gear 246 disposed on the mounting plate 222 of the mounting frame 22 and engaged with the worm 244; a first coaxial gear 248 arranged on the first transmission gear 246; a second transmission gear 241 disposed on the mounting plate 222 and engaged with the first coaxial gear 248; a second coaxial gear 243 arranged on the second transmission gear 241; a third transmission gear 245 disposed on the mounting plate 222 and engaged with the second coaxial gear 243; a third coaxial gear 247 arranged on the third transmission gear 245; and a rack 249 engaged with the third coaxial gear 247 and arranged in a vertical direction.

The liftable aroma diffuser body 10 is configured to atomize and diffuse essential oil into the surrounding environment. It comprises a sliding cylinder 12 slidably arranged within the guide sleeve 40, and an atomizing module 14 disposed inside the sliding cylinder 12.

The atomizing module 14 comprises: an essential oil bottle 142; an oil tube 144 inserted into the essential oil bottle 142; an atomizing core body 146 mounted at the mouth of the essential oil bottle 142; an atomizing core 148 disposed inside the atomizing core body 146 and in fluid communication with the oil tube 144; an atomizing head 141 sleeved over the atomizing core body 146; an atomizing head lower cover 143 disposed on the top of the atomizing head 141; an oil baffle lower cover 145 and an oil baffle upper cover 147, which are engaged with each other and mounted on the atomizing head lower cover 143; and an atomizing head upper cover 149 disposed on the top of the oil baffle upper cover 147.

Preferably, an air pump 30 is disposed on the surface of the mounting plate 222 opposite to the surface on which the transmission module 24 is mounted. The air pump 30 provides high-pressure gas to the atomizing assembly inside the liftable aroma diffuser body 10, so as to atomize and spray the essential oil from the essential oil bottle. Preferably, the air pump 30 is fixed to the mounting plate 222 by means of a pair of anti-vibration mounting sleeves 32. Preferably, the air pump 30 is hermetically connected to the atomizing head 141 through an air tube 36. The essential oil in the essential oil bottle 142 enters the atomizing core 148 through the oil tube 144, while the air pump 30 delivers gas to the atomizing head 141, causing the essential oil to be dispersed and atomized by the gas and then emitted outward through the atomizing head upper cover 149.

Preferably, a guide rail 122 is disposed on an outer sidewall of the sliding cylinder 12; and a guide groove 42 is formed on an inner sidewall of the guide sleeve 40. The guide rail 122 is slidably fitted within the guide groove 42, allowing the sliding cylinder 12 to move smoothly up and down within the guide sleeve 40. The top of the rack 249 is fixedly connected to the lifting platform 26, so that when the transmission module 24 operates, the lifting platform 26 is driven to move synchronously upward and downward. The guide sleeve 40 is disposed on the support plate 226, and the liftable aroma diffuser body 10 is sleeved within the guide sleeve 40 and fixed to the lifting platform 26.

Preferably, the transmission module 24 further includes a limit switch 2491 disposed on the mounting plate 222 below the lower end of the rack 249. When the rack 249 moves downward to a certain position, the lower end of the rack 249 contacts the limit switch 2491, thereby stopping the operation of the motor 242. This indicates that the liftable aroma diffuser body 10 has descended to a predetermined position, and the motor then ceases operation.

Preferably, the rack 249 is longitudinally provided with a guide track 2492; the mounting plate 222 is longitudinally provided with a guide groove 2222; and the guide track 2492 is slidably fitted within the guide groove 2222. This configuration ensures that the rack 249 always moves vertically upward and downward, thereby ensuring that the liftable aroma diffuser body 10 also moves vertically within the guide sleeve 40. Consequently, smooth vertical movement is maintained, and friction between the liftable aroma diffuser body 10 and the guide sleeve 40 is reduced. More preferably, the support plate 226 is provided with an opening 2262 through which the rack 249 passes and is fixed to the lifting platform 26.

Further preferably, a pull rod 123 is disposed at the bottom of the lifting platform 26, and the end of the pull rod 123 is provided with a limiting ring 125. The limiting ring 125 is fixed to the pull rod 123 by means of a nut (not labeled). The support plate 226 is further provided with a through hole 2264 for the pull rod 123 to pass through. The purpose of providing the pull rod and limiting ring is as follows: when the transmission module 24 drives the rack 249 to move upward relative to the guide sleeve 40, the liftable aroma diffuser body 10 moves upward synchronously with the rack 249. When the liftable aroma diffuser body 10 has been raised such that its essential oil bottle is just exposed from the top of the guide sleeve 40, this position is sufficient for the user to conveniently replace the essential oil bottle, and there is no need for further upward movement. Therefore, the limiting ring abuts against the bottom of the guide sleeve 40 to restrict further upward movement of the liftable aroma diffuser body 10. When the motor 242 detects the resistance caused by the limiting ring, it automatically stops operation, thereby preventing damage to the motor, transmission gears, and rack.

More preferably, a protective cover 249222 is disposed on the mounting plate 222 to enclose the transmission module 24, thereby providing protection for the transmission module 24.

In the present application, by integrating the atomizing module inside a sliding cylinder, which is slidably arranged within the guide sleeve, the sliding cylinder and the atomizing module therein are driven by the lifting drive mechanism to move upward relative to the guide sleeve. This configuration facilitates the upward movement of the essential oil bottle, so that it is exposed from the guide sleeve, enabling convenient and quick replacement of the essential oil bottle.

After replacement, the lifting drive mechanism drives the sliding cylinder and the atomizing module therein to retract downward into the guide sleeve, thereby completing the bottle replacement process. Compared with the prior art, the integrated aroma diffuser provided by the present application allows extremely convenient and quick replacement of the essential oil bottle during use, which undoubtedly enhances user experience significantly. Meanwhile, the overall structure is simple, and the production cost is low.

Preferably, an open spiral oil-baffle wall 1472 and an annular retaining wall 1474 are formed on the oil baffle upper cover 147. The arrangement of the open spiral oil-baffle wall 1472 and the annular retaining wall 1474 enables the flow path of the atomized essential oil particles to become more tortuous, thereby reducing the noise generated during airflow and providing a better user experience.

Preferably, a positioning rib 1412 and a positioning plane 1414 are defined on the outer sidewall of the atomizing head 141; a corresponding positioning groove 124 and positioning wall 126 are provided on the inner sidewall of the sliding cylinder 12. The positioning rib 1412 is clamped within the positioning groove 124, and the positioning plane 1414 abuts against the positioning wall 126. The cooperation of these two structures provides an anti-misplacement function. That is, when the atomizing module 14 is inserted into the sliding cylinder 12, it can only be inserted in a fixed direction, ensuring smooth alignment and proper fitting of all components.

Preferably, the integrated aroma diffuser 100 further comprises a housing 60, inside which the liftable aroma diffuser body 10, the lifting drive mechanism 20, the air pump 30, and the guide sleeve 40 are arranged. In this way, all these components are enclosed within the housing 60, while the top portion of the liftable aroma diffuser body 10 protrudes from the housing 60, allowing it to move vertically relative to the housing 60 and the guide sleeve 40. After assembly, the overall appearance of the aroma diffuser product is improved, providing the user with a more pleasing visual experience.

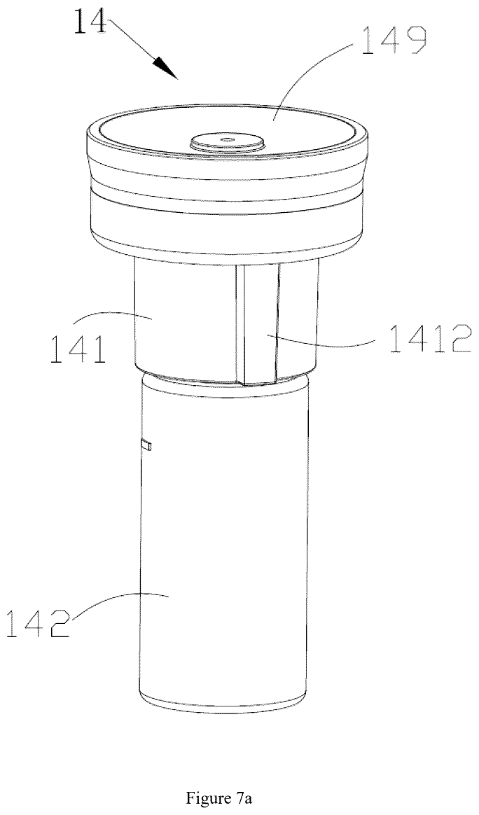

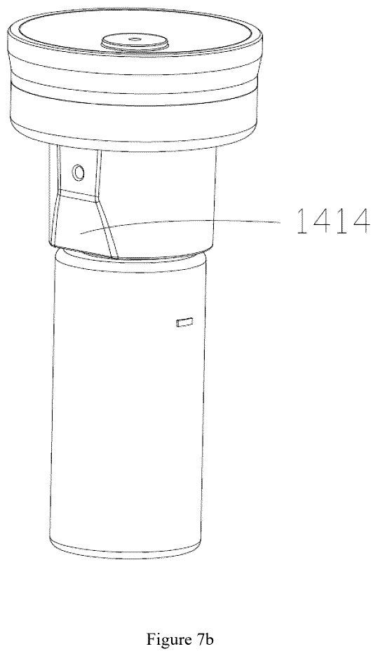

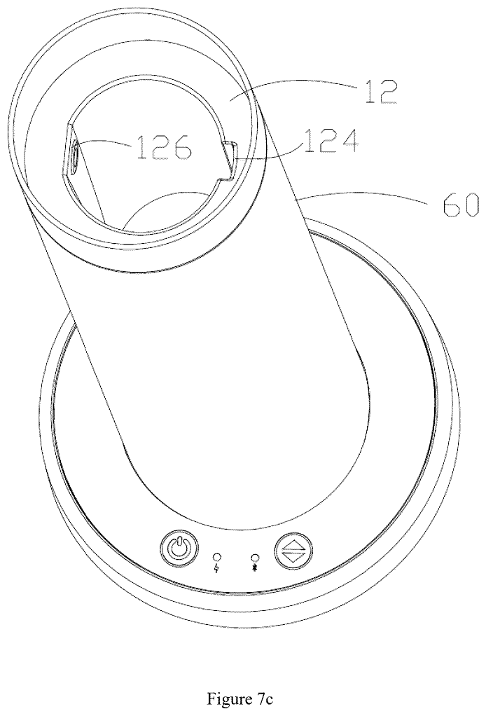

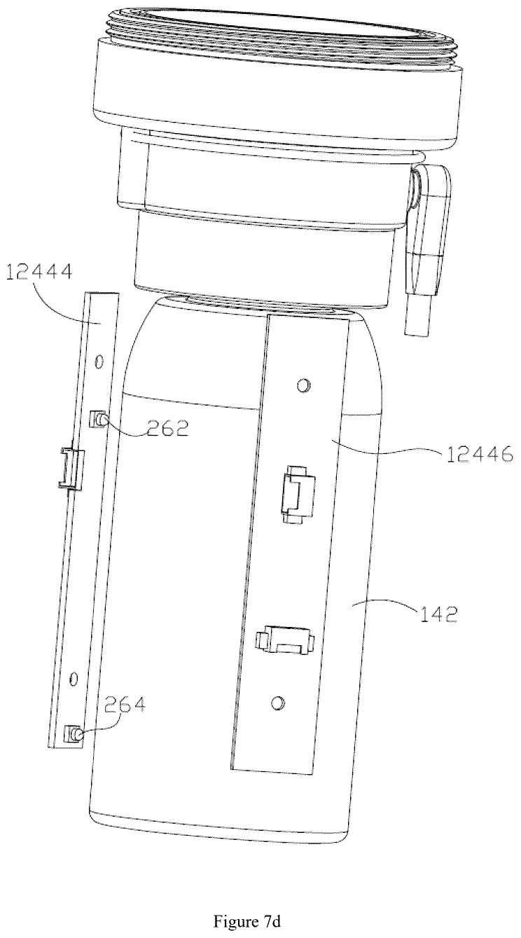

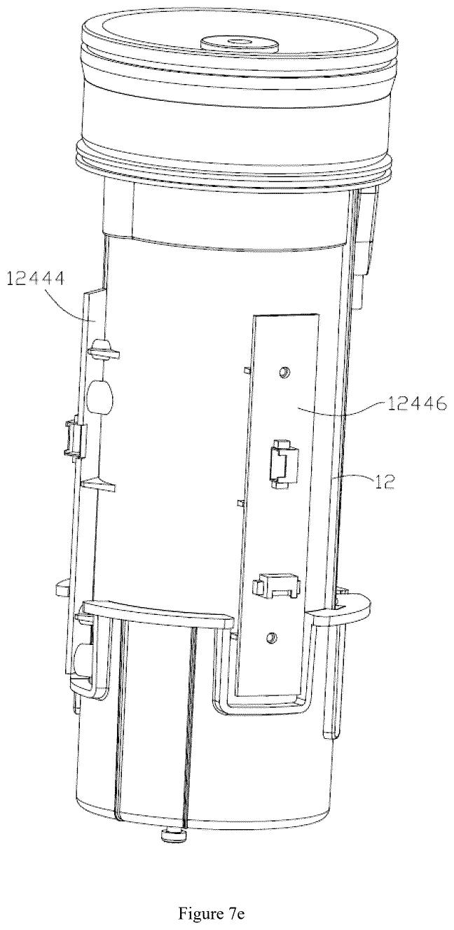

Preferably, and referring to FIG. 7d-7e, a first essential-oil level detection circuit board 12444 and a corresponding second essential-oil level detection circuit board 12446 are provided on the sidewall or bottom wall of the sliding cylinder 12. A high-level emitter 262 is provided at the top of the first essential-oil level detection circuit board 12444, and a low-level emitter 264 is provided at its bottom. Correspondingly, a high-level receiver (not shown) is provided at the top of the second essential-oil level detection circuit board 12446, and a low-level receiver (not shown) is provided at its bottom.

The corresponding emitters emit light, while the receivers receive the emitted light. Under normal conditions, when the essential oil inside the essential oil bottle 142 is sufficient, the oil blocks the light transmission path between the high-level emitter and the high-level receiver, preventing the receiver from receiving light.

When the essential oil in the bottle 142 is consumed to a certain level, i.e., when the oil surface falls below the position of the high-level emitter, the light path between the high-level emitter and the high-level receiver is no longer blocked by the oil, so the receiver receives light and triggers the second essential-oil level detection circuit board 12446 to generate a corresponding alarm signal, notifying the user that the oil level has dropped.

Similarly, when the essential oil is nearly depleted, i.e., when the oil surface falls below the position of the low-level emitter, the light paths between both the low-level emitter and receiver, as well as the high-level emitter and receiver, become unblocked. The receivers therefore receive light, triggering the second essential-oil level detection circuit board 12446 to generate an alarm signal, indicating that the essential oil is almost exhausted and needs replenishment. The system may alert the user to replace the essential oil bottle through various methods, such as sound, light, or a combination thereof.

The upper cover of the atomizing head and the lower cover of the oil baffle in the present application may also be provided with a structure that prevents the leakage of essential oil from the atomizing head upper cover when the entire aroma diffuser is accidentally overturned.

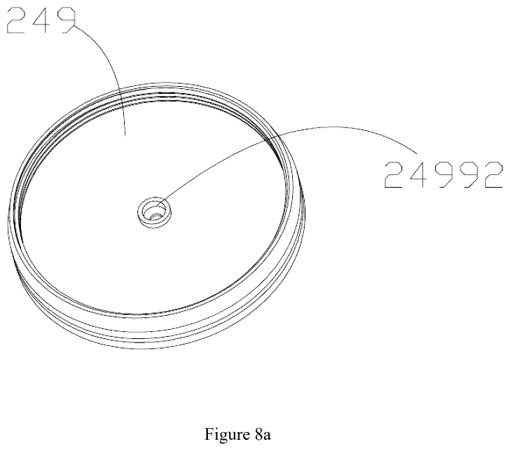

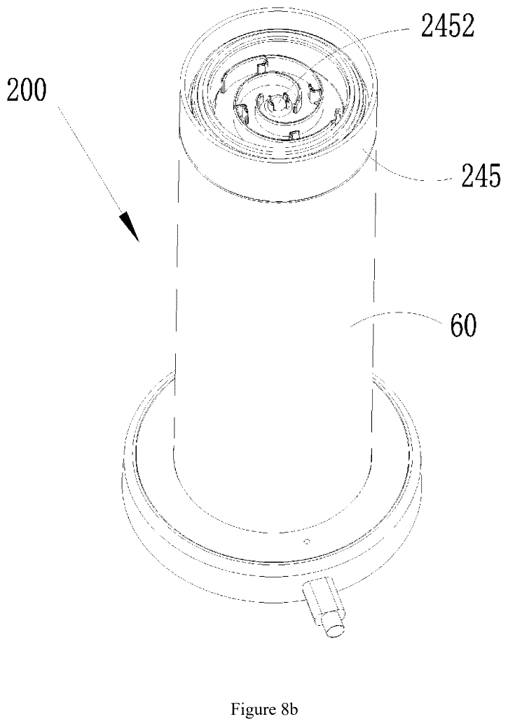

Specifically, according to another embodiment of the present application, and referring to FIG. 8a-8b, 9, and 12, an aroma diffuser 200 is provided, in which an atomizing head upper cover 249 is formed with an atomizing outlet hole 24992. Similarly, a mist outlet 2452 is formed on the oil baffle lower cover 245 located at the top of the housing 60. A steel ball 202 is movably disposed between the atomizing outlet hole 24992 and the mist outlet 2452.

In particular, the bottom of the atomizing outlet hole 24992 forms a conical structure. When the integrated aroma diffuser is accidentally knocked over and laid flat, or when the user unintentionally inverts the aroma diffuser, the steel ball automatically slides, under the influence of gravity, into the conical structure of the atomizing outlet hole, thereby sealing it.

In this manner, the essential oil leaking from the oil baffle lower cover cannot seep through the atomizing outlet hole to the exterior of the aroma diffuser. Thus, not only is the waste of essential oil prevented, but the cleanliness of the entire aroma diffuser is also maintained.

The integrated aroma diffuser may further comprise a main power board provided with a tilt alarm device. The tilt alarm device may be configured to issue an alarm signal when the inclination angle of the integrated aroma diffuser relative to the horizontal plane is not less than 45 degrees, thereby alerting the user that the aroma diffuser is in a near-tipping position.

Furthermore, during the use and sale of the aroma diffuser, in order to prevent unscrupulous merchants from opening the essential oil bottle and replacing the essential oil with inferior oil, the essential oil bottle and the atomizing head are designed with a tamper-proof structure.

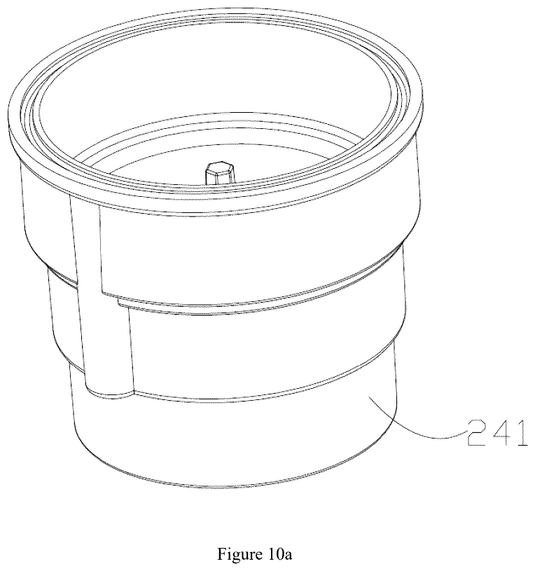

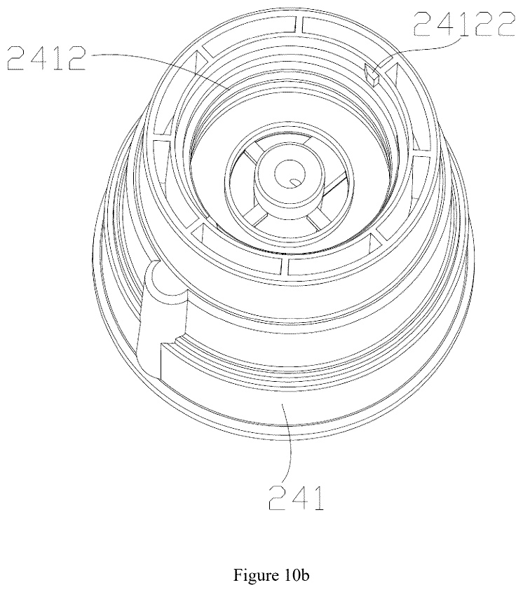

Specifically, referring to FIG. 10a-10b, 11, and 12, the atomizing head 241 has an internal threaded section 2412, and the top of the internal threaded section 2412 is provided with a first barb 24122. Correspondingly, the essential oil bottle 242 comprises a bottle body 2422 and a bottle neck 2424 positioned at the top of the bottle body 2422. The outer surface of the bottle neck 2424 is provided with an external threaded section 24242, and the bottom of the external threaded section 24242 is provided with a second barb 24244, which cooperates with the first barb 24122.

When the atomizing head is fastened to the essential oil bottle by threading, as the rotation approaches completion, the first barb gradually elastically passes over the second barb and, due to its elasticity, becomes hooked with the second barb. Thus, when the user attempts to rotate the atomizing head in the reverse direction, the two barbs interlock or abut against each other, preventing reverse rotation. As a result, the atomizing head cannot be removed in the reverse direction to replace the essential oil, thereby preventing unscrupulous merchants from secretly refilling the bottle with inferior essential oil to deceive consumers.

What is claimed is:

1. An integrated aroma diffuser, comprising:
a liftable aroma diffuser body slidably disposed within a guide sleeve;
a lifting drive mechanism configured to drive the liftable aroma diffuser body to move vertically relative to the guide sleeve, the lifting drive mechanism including a mounting frame, a transmission module disposed on the mounting frame, and a lifting platform driven by the transmission module;
wherein the liftable aroma diffuser body is mounted on the lifting platform; and
the mounting frame includes a mounting plate, a plurality of supporting columns provided around a periphery of the mounting plate, and a support plate disposed at top ends of the supporting columns.

2. The integrated aroma diffuser according to claim 1, wherein the transmission module comprises:
a motor disposed on the mounting plate and having an output shaft;
a worm disposed on the output shaft;
a first transmission gear disposed on the mounting plate and engaged with the worm;
a first coaxial gear disposed on the first transmission gear;
a second transmission gear disposed on the mounting plate and engaged with the first coaxial gear;
a second coaxial gear disposed on the second transmission gear;
a third transmission gear disposed on the mounting plate and engaged with the second coaxial gear;
a third coaxial gear disposed on the third transmission gear;
and a rack engaged with the third coaxial gear and arranged vertically.

3. The integrated aroma diffuser according to claim 2, wherein the liftable aroma diffuser body comprises a sliding cylinder slidably disposed within the guide sleeve and an atomizing module disposed within the sliding cylinder.

4. The integrated aroma diffuser according to claim 3, wherein the atomizing module comprises:
an essential oil bottle;
an oil tube inserted into the essential oil bottle;
an atomizing core body installed at the mouth of the essential oil bottle;
an atomizing core disposed within the atomizing core body and in fluid communication with the oil tube;
an atomizing head sleeved on the atomizing core body;
an atomizing head lower cover disposed at the top of the atomizing head;
an oil baffle lower cover and an oil baffle upper cover, which are engaged with each other and disposed on the atomizing head lower cover;
and an atomizing head upper cover disposed on the oil baffle upper cover.

5. The integrated aroma diffuser according to claim 4, wherein an air pump is disposed on the surface of the mounting plate opposite to the surface on which the transmission module is mounted;
the air pump is fixed to the mounting plate by a pair of anti-vibration mounting sleeves;
and the air pump is hermetically connected to the atomizing head through an air tube.

6. The integrated aroma diffuser according to claim 3, wherein a guide rail is provided on the outer sidewall of the sliding cylinder; a guide groove is formed on the inner sidewall of the guide sleeve; and the guide rail is slidably fitted within the guide groove.

7. The integrated aroma diffuser according to claim 5, wherein the top of the rack is fixedly connected to the lifting platform; the guide sleeve is disposed on the support plate; and the liftable aroma diffuser body is sleeved within the guide sleeve and fixed to the lifting platform.

8. The integrated aroma diffuser according to claim 7, wherein the transmission module further comprises a limit switch disposed on the mounting plate and positioned below the lower end of the rack.

9. The integrated aroma diffuser according to claim 8, wherein a guide track is longitudinally provided on the rack; a guide groove is longitudinally formed on the mounting plate; and the guide track is slidably fitted within the guide groove.

10. The integrated aroma diffuser according to claim 9, wherein a pull rod is disposed at the bottom of the lifting platform, and a limiting ring is formed at the end of the pull rod; the limiting ring is fixed to the end of the pull rod by means of a nut; and the support plate is further provided with a through hole through which the pull rod passes.

11. The integrated aroma diffuser according to claim 9, wherein positioning ribs and a positioning plane are formed on the outer sidewall of the atomizing head; a positioning groove and a positioning wall are provided on the inner sidewall of the sliding cylinder; the positioning ribs are clamped within the positioning grooves, and the positioning plane abuts against the positioning wall.

12. The integrated aroma diffuser according to claim 6, wherein a first essential-oil level detection circuit board and a corresponding second essential-oil level detection circuit board are provided on the sidewall of the sliding cylinder; a high-level emitter is disposed at the top of the first essential-oil level detection circuit board, and a low-level emitter is disposed at its bottom; a high-level receiver is disposed at the top of the second essential-oil level detection circuit board, and a low-level receiver is disposed at its bottom.

13. The integrated aroma diffuser according to claim 4, wherein an atomizing outlet hole is formed on the atomizing head upper cover; a mist outlet is formed on the oil baffle lower cover; a steel ball is movably disposed between the atomizing outlet hole and the mist outlet; and the aroma diffuser further comprises a main power board provided with a tilt alarm device, wherein the tilt alarm device is configured to issue an alarm signal when the inclination angle of the integrated aroma diffuser relative to the horizontal plane is not less than 45 degrees.

14. The integrated aroma diffuser according to claim 4, wherein the interior of the atomizing head is provided with an internal threaded section, and a first barb is provided at the top of the internal threaded section; the essential oil bottle comprises a bottle body and a bottle neck located at the top of the bottle body; the outer surface of the bottle neck is provided with an external threaded section, and a second barb, which cooperates with the first barb, is provided at the bottom of the external threaded section.
